(19) United States
(12) Patent Application Publication
(10) Pub. No.: US 2024/0099930 A1
(43) Pub. Date: Mar. 28, 2024

(54) MULTIFUNCTIONAL MASSAGE DEVICE

(71) Applicant: Dongguan Duoguo Zhichuang Technology Co., Ltd., Dongguan City (CN)

(72) Inventor: Changzhi LUO, Dongguan City (CN)

(21) Appl. No.: 18/533,725

(22) Filed: Dec. 8, 2023

(30) Foreign Application Priority Data
Jul. 4, 2023 (CN) 2023108131344

(51) Int. Cl. A61H 19/00

(52) U.S. Cl. CPC A61H 19/32; A61H 2201/1207

(57) ABSTRACT

The disclosure provides a multifunctional massage device, wherein a driving device, an air extraction pump and an electric control valve are mounted in a mounting cavity of a housing, and a flexible massage cylinder is placed in a massage cavity of the housing; an air extraction port of the air extraction pump is connected to an air outlet of the electric control valve through a pipeline, and an air inlet of the electric control valve is connected to an air outlet of the flexible massage cylinder through a pipeline; and the driving device drives a telescopic member to perform telescopic rotation motion, and the flexible massage cylinder is connected to the telescopic member. The present invention achieves the effects of tightening-releasing type massage and friction type massage on one message device.

### TECHNICAL FIELD

The present invention relates to the technical field of physical therapy devices, and in particular, to a multifunctional massage device.

### BACKGROUND

The massage device is widely applied and popularized based on the remarkable effect of the massage device. At present, most massage devices use an automatic beating type. The beating type massage achieves the effect of massaging the body by driving a massage component to perform telescopic reciprocating motion to repeatedly impact a part of the human body that needs to be massaged. However, the massage device has poor comfort during massage and a single function.

### SUMMARY

The present invention is intended to provide a multifunctional massage device designed to solve the above technical defects.

The multifunctional massage device designed by the present invention comprises: a housing, wherein the housing is provided with a mounting cavity and a massage cavity, a mounting member, a driving device, an air extraction pump and an electric control valve are mounted in the mounting cavity, a mounting cylinder and a flexible massage cylinder are placed in the massage cavity, an opening of the flexible massage cylinder faces an opening of the massage cavity, and the flexible massage cylinder is mounted in a cylinder cavity of the mounting cylinder; an electric heating sheet is bonded on an inner wall of the cylinder cavity of the mounting cylinder, or a vibrator is embedded in the flexible massage cylinder; the driving device is mounted on the mounting member;

the driving device comprises a motor, a fixed cylinder, a gear set, a reciprocating screw, a telescopic member and a crescent arc guide member, the fixed cylinder is provided with a guide channel arranged along a length direction of the fixed cylinder, the reciprocating screw penetrates through the guide channel, a rotating shaft of the motor is in transmission connection with a driving end of the reciprocating screw through the gear set, a telescopic end of the reciprocating screw is fixed with a telescopic shaft, a driving end of the telescopic shaft passes through a closed end of the fixed cylinder and then is connected to the telescopic member, two mutually crossed and communicated spiral grooves are formed on the reciprocating screw, the arc guide member is positioned in the spiral groove, and a shaft body of the arc guide member is rotatably mounted in a shaft hole on an inner wall of the guide channel;

the mounting member is provided with an input through hole and an air extraction through hole, an air extraction port of the air extraction pump is connected to and communicated with the air extraction through hole through a pipeline, an air inlet of the electric control valve is connected to and communicated with the input through hole through a pipeline, and a flexible ring is mounted at a port of the housing; and

a central mounting part of the mounting cylinder is mounted on the telescopic member, a conductive structure is provided between the telescopic member and the central mounting part of the mounting cylinder, and the conductive structure is electrically connected to the vibrator or the electric heating sheet through a conductive wire.

The multifunctional massage device designed by the present invention has the following beneficial effects.

1. During massage, a to-be-massaged part is placed in an inner cavity of a flexible massage cylinder, and flexible protrusions on the inner wall of the flexible massage cylinder abut against an outer wall of the to-be-massaged part under an elastic action of the flexible massage cylinder; in this case, a driving device is used to drive a telescopic member and the flexible massage cylinder to perform telescopic motion so as to perform friction type massage on the to-be-massaged part, or an air extraction pump and an electric control valve are started to perform air extraction on the flexible massage cylinder, so that the flexible massage cylinder tightens the to-be-massaged part for a period of time and then releases the to-be-massaged part, wherein the releasing is closed by an electromagnetic valve, allowing outside air to enter the flexible massage cylinder, and the tightening is conducted by the electric control valve; therefore, during the tightening-releasing type massage or friction type massage, the vibrator or electric heating sheet can be started to provide heating or vibration massage assistance during the massage process to improve the massage effect. In addition, the present invention achieves tightening-releasing type massage, friction type massage, vibration massage or heating massage on one massage device, so that the massage method can achieve diversified effects.

2. The telescopic member is connected to the sleeve body in a buckling mode, so that the assembly and disassembly are convenient.

### BRIEF DESCRIPTION OF DRAWINGS

FIG. 1 is a schematic diagram (I) of an overall structure;

FIG. 2 is a schematic diagram (II) of an overall structure;

FIG. 3 is an enlarged view at A;

FIG. 4 is a schematic diagram of a partial structure;

FIG. 5 is a schematic diagram (I) of a conductive structure;

FIG. 6 is a schematic diagram (II) of a conductive structure;

FIG. 7 is a schematic diagram of a structure of a first connecting body;

FIG. 8 is a schematic diagram of a structure of a third connecting body;

FIG. 9 is a schematic diagram (I) of a structure of the combination of a mounting cylinder and a flexible massage cylinder;

FIG. 10 is a schematic diagram (II) of a structure of the combination of a mounting cylinder and a flexible massage cylinder;

FIG. 11 is a schematic diagram (III) of a structure of the combination of a mounting cylinder and a flexible massage cylinder;

FIG. 12 is a schematic diagram (I) of the combination of a positioning cylinder and a fixed frame;

FIG. 13 is a schematic diagram (II) of the combination of a positioning cylinder and a fixed frame;

FIG. 14 is a schematic diagram of a structure of a gear set;

FIG. 15 is a schematic diagram (I) of a structure of a driving device;

FIG. 16 is a schematic diagram (II) of a structure of a driving device; and

FIG. 17 is a schematic diagram (III) of a structure of a driving device.

In the drawings, 1: housing; 10: flexible ring; 11: first cylinder body; 111: massage cavity; 112: mounting cylinder; 113: flexible massage cylinder; 114: flexible protrusion; 115: vibrator; 116: central mounting part; 117: sleeve body; 118: buckle groove; 119: limiting protrusion; 12: second cylinder body; 120: controller; 121: mounting cavity; 13: telescopic member; 131: telescopic disk; 132: telescopic sleeve; 133: gap; 134: elastic buckle; 135: limiting recess; 14: conductive structure; 141: first connecting body; 142: second connecting body; 143: third connecting body; 101: first conductive part; 102: second conductive part; 103: third conductive part; 104: fourth conductive part; 105: fifth conductive part; 106: sixth conductive part; 107: seventh conductive part; 108: eighth conductive part; 109: ninth conductive part; 15: driving device; 151: fixed cylinder; 152: guide channel; 153: reciprocating screw; 154: gear set; 155: telescopic shaft; 156: guide rod; 157: motor; 158: driving gear; 159: first driven gear; 160: second driven gear; 161: third driven gear; 162: protective casing; 163: protective cover; 164: spiral groove; 165: arc guide member; 166: shaft hole; 167: shaft body; 168: half cylinder; 16: air extraction pump; 17: electric control valve; 18: positioning cylinder; 19: fixed frame; 2: end cover; 181: input through hole; 182: air extraction through hole.

### DETAILED DESCRIPTION OF EMBODIMENTS

The following clearly and completely describes the technical solutions in embodiments of the present invention with reference to the accompanying drawings in embodiments of the present invention. It is clear that the described embodiments are merely a part rather than all of embodiments of the present invention. Based on the embodiments of the present invention, all other embodiments obtained by those of ordinary skill in the art fall within the protection scope of the present invention.

### EMBODIMENT

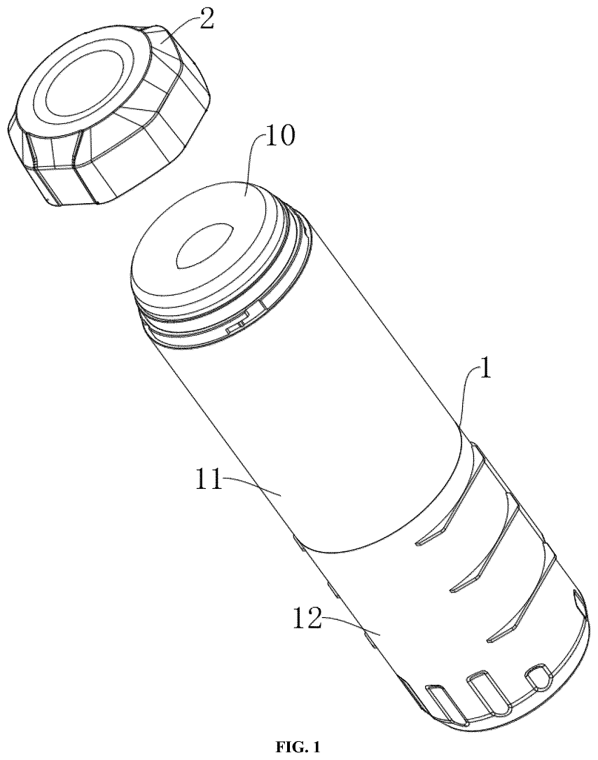

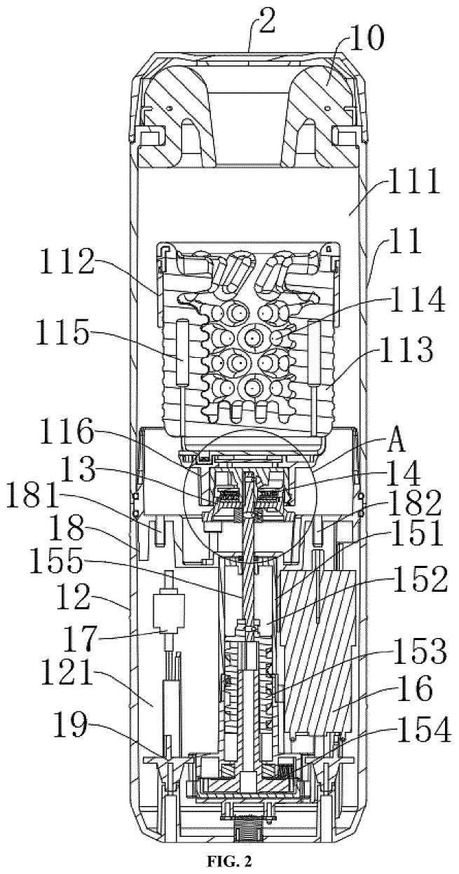

As shown in FIGS. 1 and 2, the multifunctional massage device described in this embodiment is generally used for finger massage and massages fingers that are frequently not active due to hand diseases to promote blood circulation. In addition, the multifunctional massage device is provided to match with the fingers and can also be used for massaging male sexual organs.

As shown in FIGS. 2 and 11, the multifunctional massage device comprises a housing 1, wherein the housing 1 comprises a first cylinder 11 and a second cylinder 12, a mounting cavity 121 is provided on the second cylinder 12, a massage cavity 111 is provided on the first cylinder 11, the second cylinder 12 and the first cylinder 11 are respectively sleeved at a left end and a right end of a positioning cylinder 18, a sealing ring is provided between an inner wall of the second cylinder 12 and an outer wall of the positioning cylinder 18, the second cylinder 12 is fixed on a fixed frame 19 on the positioning cylinder 18 through screws, and an inner wall of the first cylinder 11 is connected to the outer wall of the positioning cylinder 18 through a buckle for convenient disassembly and assembly. A mounting member, a driving device 15, an air extraction pump 16 and an electric control valve 17 are mounted in the mounting cavity 121, a mounting cylinder 112 and a flexible massage cylinder 113 are placed in the massage cavity 111, the driving device 15 is mounted on the mounting member, the flexible massage cylinder 113 and flexible protrusions 114 on the inner wall of the flexible message cylinder are integrally made by silica gel, the mounting cylinder 112, the second cylinder 12, the first cylinder 11, the positioning cylinder 18 and the fixed frame 19 are all made of plastic materials, an opening of the flexible massage cylinder 113 faces an opening of the massage cavity 111, the flexible massage cylinder 113 is mounted in a cylinder cavity of the mounting cylinder 112, an electric heating sheet is bonded on the inner wall of the cylinder cavity of the mounting cylinder 112, the electric heating sheet can be a flexible electric heating sheet formed by a graphene electric heating sheet or a heating copper wire, a vibrator 115 is embedded in the flexible massage cylinder 113, the vibrator 115 is driven by a motor 157 to rotate an eccentric block to generate vibration, an inner cavity of the flexible massage cylinder 113 is matched with fingers and has elasticity so as to adapt to fingers with different thicknesses, the flexible massage cylinder 113 and the massage cavity 111 are arranged in a tight fit mode, or the inner cavity of the flexible massage cylinder 113 can also be arranged to be matched with male sexual organs.

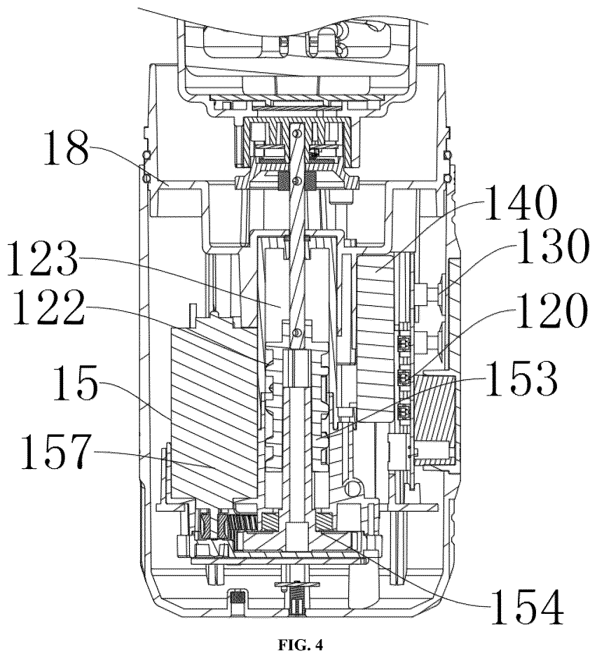

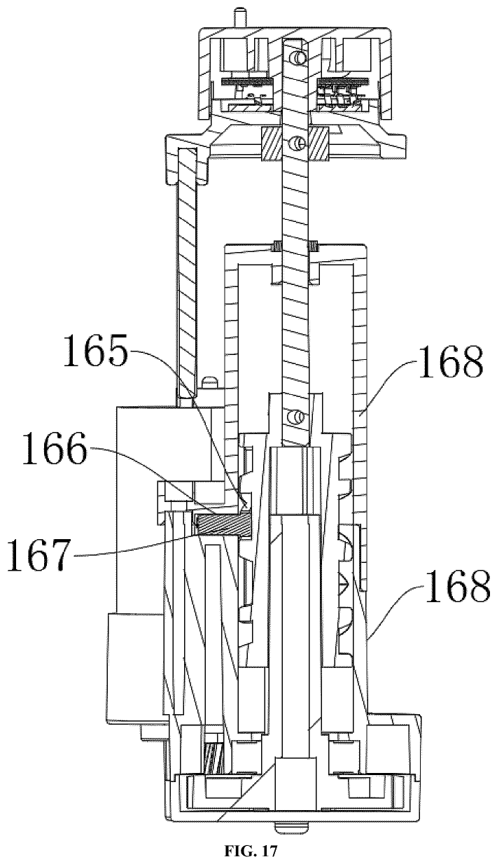

As shown in FIGS. 2, 4, 12, 14, 15, 16 and 17, the driving device 15 comprises a motor 157, a fixed cylinder 151, a gear set 154, a reciprocating screw 153, a telescopic member 13 and a crescent arc guide member 165, the fixed cylinder 151 is provided with a guide channel 152 arranged along a length direction of the fixed cylinder, the reciprocating screw 153 penetrates through the guide channel 152, a rotating shaft of the motor 157 is in transmission connection with a driving end of the reciprocating screw 153 through the gear set 154, a telescopic end of the reciprocating screw 153 is fixed with a telescopic shaft 155, a driving end of the telescopic shaft 155 passes through a closed end of the fixed cylinder 151 and then is connected to the telescopic member 13, two mutually crossed and communicated spiral grooves 164 are formed on the reciprocating screw 153, that is, middle parts of the two spiral grooves 164 are crossed and communicated, and the two ends thereof are also communicated with each other; the arc guide member 165 is positioned in the spiral groove 164, and a shaft body 167 of the arc guide member 165 is rotatably mounted in a shaft hole 166 on an inner wall of the guide channel 152; after the motor 157 drives the reciprocating screw 153 to rotate, under the relative action of the arc guide member 165 and the continuous tracks provided by the two connected spiral grooves 164, the reciprocating screw 153 performs the telescopic motion to drive the telescopic shaft 155 and the telescopic member to perform the telescopic motion, and the fixed cylinder 151 is fixed in the positioning cylinder 18.

As shown in FIG. 17, the fixed cylinder 151 is formed by assembling two half cylinders 168 to form the guide channel 152, and two semicircular grooves are provided at a side of an assembled part of the two half cylinders 168; and the two semicircular grooves are symmetrically assembled to form the shaft hole 166, and the two half cylinders 168 are fixed to each other.

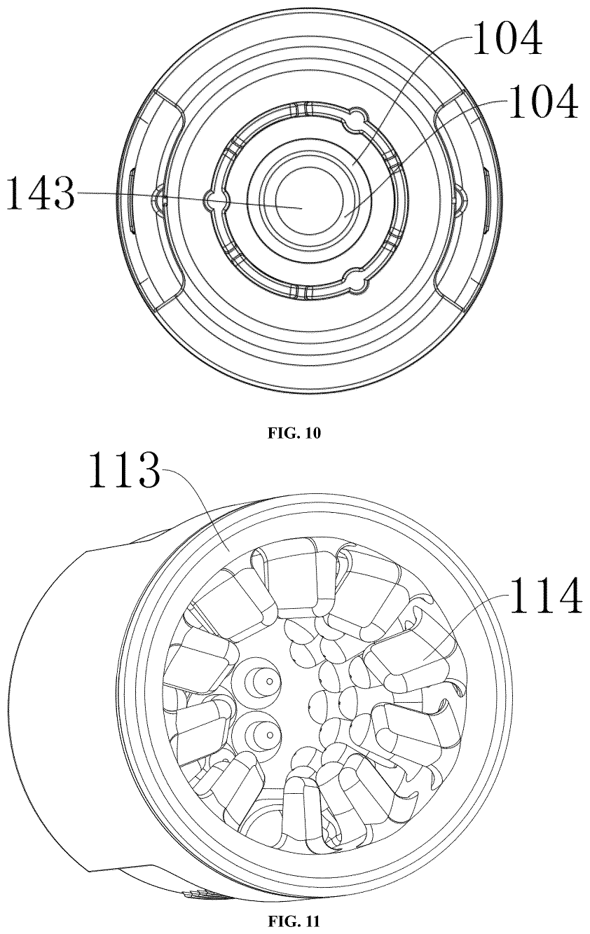

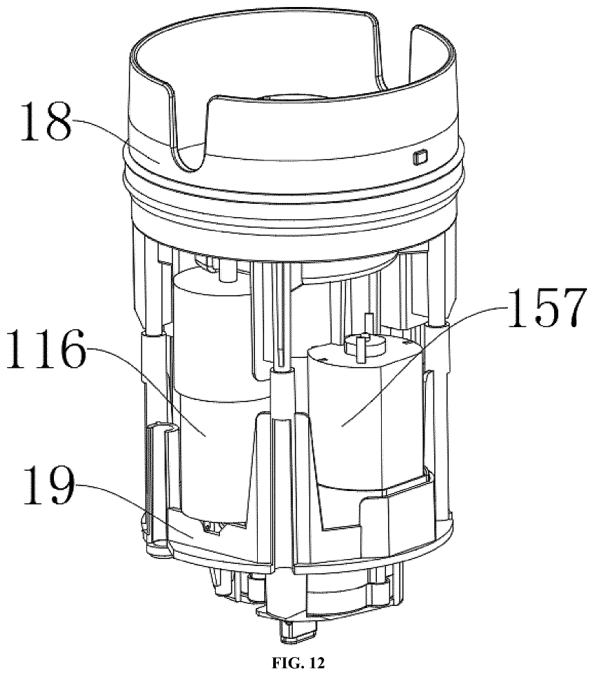

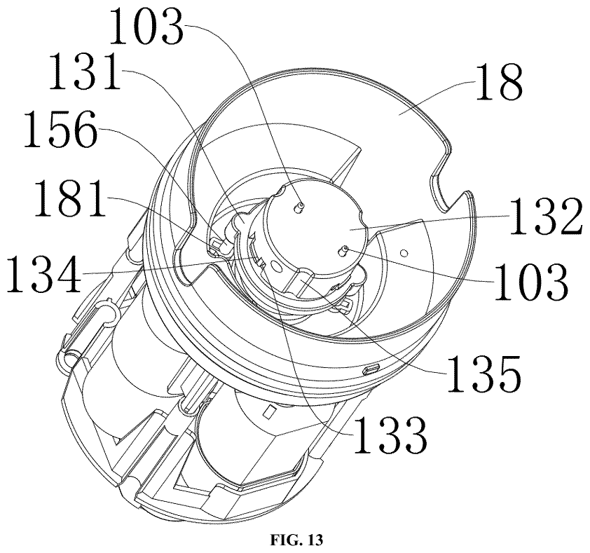

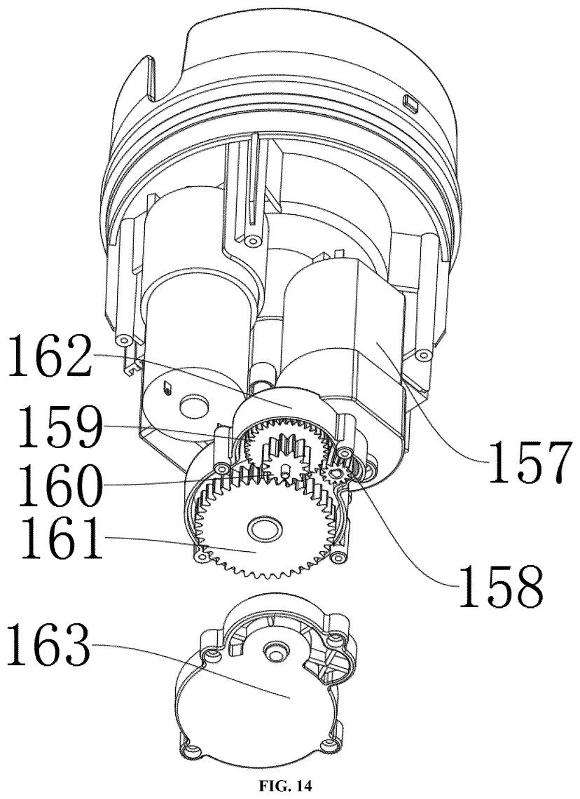

As shown in FIGS. 2, 3, 9 and 13, the central mounting part 116 of the mounting cylinder 112 is mounted on the telescopic member 13, the telescopic member 13 comprises a telescopic disk 131 and a telescopic sleeve 132, the telescopic sleeve 132 is sleeved on a positioning ring of the telescopic disk 132 and forms a cavity, the first connecting body 141 and the second connecting body 142 are fixed in the cavity, the driving end of the telescopic shaft 155 is connected to the telescopic disk 131 or the telescopic sleeve 132 after penetrating through the telescopic disk 131, at least one limiting recess 135 and a plurality of spaced apart gaps 133 are formed at a peripheral side of the telescopic sleeve 132 at intervals, at least one elastic buckle 134 is formed on the telescopic sleeve 132 through the gaps 133, the central mounting part 116 of the mounting cylinder 112 is a sleeve body 117, the sleeve body 117 is sleeved on the telescopic sleeve 132, at least one limiting protrusion 119 and at least one buckle groove 118 is formed on an inner wall of the sleeve body 117, the elastic buckle 134 is correspondingly buckled in the buckle groove 118, the limiting protrusion 119 is correspondingly embedded in the limiting recess 135, the third connecting body 143 is fixed in the sleeve body 117, and the two third conductive parts 103 arranged at intervals on the second connecting body 142 abut against the two fourth conductive parts 104 on the third connecting body 143 after penetrating through the telescopic sleeve 132, so that this structure is convenient to disassemble and assemble.

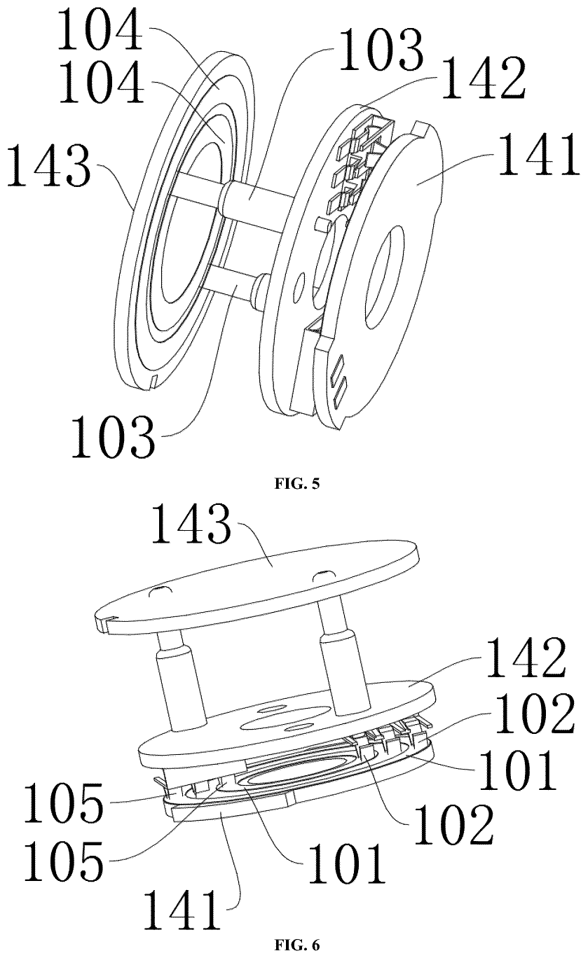

As shown in FIGS. 2, 3, 5 and 6, a conductive structure 14 is provided between the telescopic member 13 and the central mounting part 116 of the mounting cylinder 112, the conductive structure 14 is electrically connected to the vibrator 115 or the electric heating sheet through a conductive wire, the conductive structure 14 comprises a first connecting body 141, a second connecting body 142 and a third connecting body 143, the second connecting body 142 is positioned between the first connecting body 141 and the third connecting body 143, the first connecting body 141 and the second connecting body 142 are mounted on the telescopic member 13, the third connecting body 143 is fixed on the central mounting part 116 of the mounting cylinder 112, two first conductive parts 101 arranged at intervals on the first connecting body 141 respectively abut against two second conductive parts 102 arranged at intervals on the second connecting body 142, two third conductive parts 103 arranged at intervals on the second connecting body 142 respectively abut against two fourth conductive parts 104 on the third connecting body 143, the second conductive parts 102 of the second connecting body 142 are separately connected to two third conductive parts 103 through a conductive circuit, two third conductive parts 103 on the second connecting body 142 are respectively a positive pole and a negative pole, two fourth conductive parts 104 on the third connecting body 143 are respectively connected to a positive electrode and a negative electrode of a motor of the vibrator 115, and the two first conductive parts 101 on the first connecting body 141 are both annular conductive contact pieces and are concentrically arranged, so that the conductive structure 14 is electrically connected to the vibrator 115. The third conductive parts 103 penetrate through the telescopic sleeve 132, and the two first conductive parts 101 are both annular conductive contact pieces, so that the second connecting body 142 also rotates when the telescopic sleeve 132 rotates, and the second conductive parts 102 always contact with the first conductive parts 101.

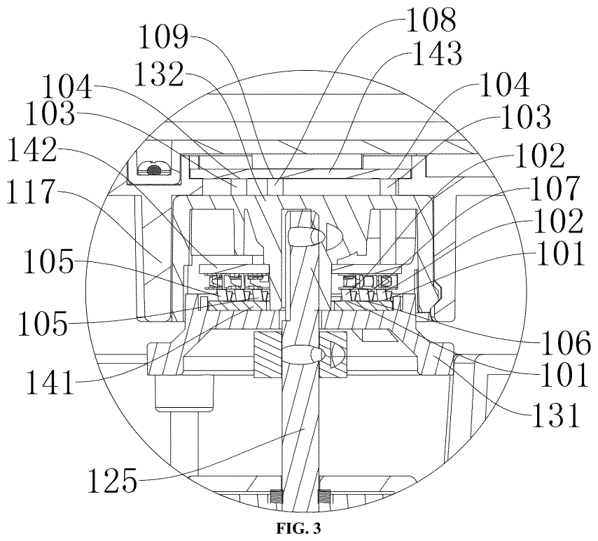

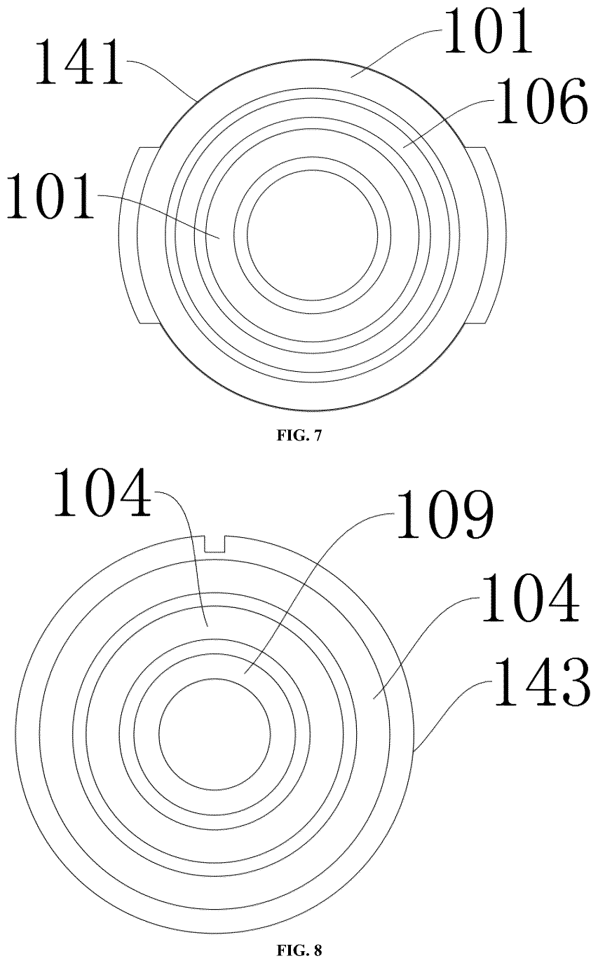

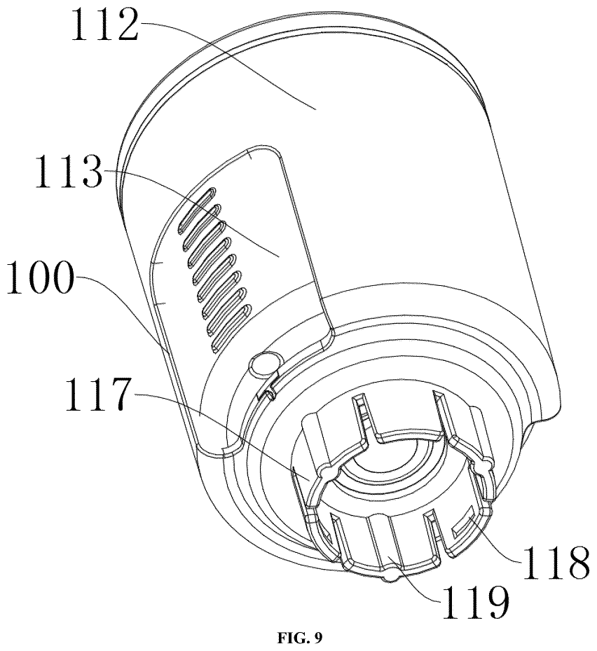

Further, as shown in FIGS. 3, 7 and 8, the first connecting body 141 is provided with a sixth conductive part 106 spaced apart from the first conductive part 101, the second connecting body 142 is further provided with a seventh conductive part 107 and an eighth conductive part 108, the seventh conductive part 107 is connected to the eighth conductive part 108 through a conductive circuit, the eighth conductive part 108 is a positive pole, the third connecting body 143 is provided with a ninth conductive part 109 spaced apart from the fourth conductive part 104, the seventh conductive part 107 abuts against the sixth conductive part 106, the eighth conductive part 108 abuts against the ninth conductive part 109 after penetrating through the telescopic sleeve 132, the ninth conductive part 109 and a fourth conductive part 104 that abuts against the third conductive part 103 of the negative pole of the third connecting body and that is on the third connecting body 143 are respectively connected to the positive electrode and the negative electrode of the electric heating sheet, and the sixth conductive part 106 is an annular conductive contact piece and is concentrically arranged with the first conductive part 101, so that the second connecting body 142 also rotates when the telescopic sleeve 132 rotates, and the seventh conductive part 107 can always contact with the sixth conductive part 106.

Preferably, as shown in FIGS. 3 and 6, two fifth conductive parts 105 arranged at intervals are further provided on the second connecting body 142, the fifth conductive parts 105 on the second connecting body 142 are separately connected to two third conductive parts 103 through a conductive circuit, the fifth conductive part 105 on the second connecting body 142 is arranged opposite to the fourth conductive part 104, two fifth conductive parts 105 abut against the two first conductive parts 101 on the first connecting body 141, and the fifth conductive part 105 is disposed at the edge of the second conductive body 142, and the fourth conductive part 104 is disposed at the edge of the third conductive body 143 so that the second connecting body 142 can stably rotate when the telescopic sleeve 132 drives the second connecting body 142 to rotate in the rotating process.

The second conductive part 102, the third conductive part 103, the fifth conductive part 105, the seventh conductive part 107 and the eighth conductive part 108 on the second connecting body 142 are conductive elastic sheets or conductive pins, the fourth conductive part 104 and the ninth conductive part 109 on the third connecting body 143 are conductive contact pieces or annular conductive contact pieces, and generally annular conductive contact pieces are used. The two annular conductive contact pieces are concentrically arranged, the centers of the two conductive elastic sheets, the centers of the two conductive pins and the centers of the two annular conductive contact pieces are arranged correspondingly, and the conductive pins are conductive elastic pins.

Certainly, a conductive elastic sheet or a conductive pin can be arranged beside the fifth conductive part 105, and the conductive elastic sheet or the conductive pin is electrically connected to the eighth conductive part 108 of the positive pole; and all of the connecting bodies are circuit boards.

As shown in FIGS. 1 and 2, the flexible ring 10 is mounted at a port of the first cylinder 11, the mounting cylinder 112 is provided with a communication port 100, the mounting member is provided with an input through hole 181 and an air extraction through hole 182, the air extraction port of the air extraction pump 16 is connected and communicated with the air extraction through hole 182 through a pipeline, and the air inlet of the electric control valve 17 is connected and communicated with the input through hole 181 through a pipeline. When negative pressure massage is performed, a to-be-massaged part is inserted into the massage cylinder 113 through the inner hole of the flexible ring 10, the inner wall of the flexible ring is in close contact with the outer wall of the to-be-massaged part, so that two ends of the inner cavity of the first cylinder are inserted into the to-be-massaged part and sealed through the mounting member and the flexible ring to form a sealed cavity; in this case, the electric control valve 17 is in a disconnected state, the air extraction pump is used to extract air to enable the sealed cavity to generate negative pressure, the negative pressure is applied to the outer wall of the massage cylinder through the communication port, and the massage cylinder is compressed and tightens the to-be-massaged part to perform negative pressure massage. After a period of massage is performed, the electric control valve 17 is conducted to enable outside air to enter the sealed cavity through the electric control valve and the input through hole 181, and a loose state of the massage cylinder is achieved, so that a user can control the massage device to perform the above operations multiple times for negative pressure massage.

The air extraction port of the air extraction pump 16 is connected to the air outlet of the electric control valve 17 through a pipeline, the air inlet of the electric control valve 17 is connected to the air outlet of the massage cylinder through a pipeline, the pipeline between the air inlet of the electric control valve 17 and the air outlet of the massage cylinder is arranged by penetrating through the positioning cylinder 18, or a connecting pipe body is provided on a partition plate of the positioning cylinder 18, and then two ends of the connecting pipe body are respectively connected to the air inlet of the electric control valve 17 and the air outlet of the massage cylinder through pipelines. When negative pressure massage is needed, the air extraction pump 16 and the electric control valve 17 are started to perform air extraction on the flexible massage cylinder 113, so that the flexible massage cylinder 113 generates negative pressure to tighten the to-be-massaged part for a period of time and then release the to-be-massaged part; and the releasing is closed by an electromagnetic valve, allowing outside air to enter the flexible massage cylinder 113, and the tightening is conducted by the electric control valve 17.

A battery and a controller 120 are also provided in the mounting cavity 121, programs of a control motor 157, the air extraction pump 16, the electric control valve 17, the vibrator 115 and the electric heating sheet are recorded in the controller 120, the battery is connected to the motor 157, the air extraction pump 16, the electric control valve 17, the vibrator 115 and the electric heating sheet separately through the controller 120 and the conductive structure 14, the electric control valve 17 uses an electromagnetic valve, and the controller 120 may be composed of an MCU chip.

Further, as shown in FIGS. 2, 4, 12 and 13, the mounting member comprises a positioning cylinder 18, the positioning cylinder 18 is positioned in the mounting cavity 121, the fixed cylinder 151 is positioned in a positioning channel of the positioning cylinder 18, the telescopic member 13 is provided with a guide rod 156, and the guide rod 156 is inserted into a guide hole of the positioning cylinder 18, so that this structural configuration enables the telescopic member 13 to move more stably and reliably.

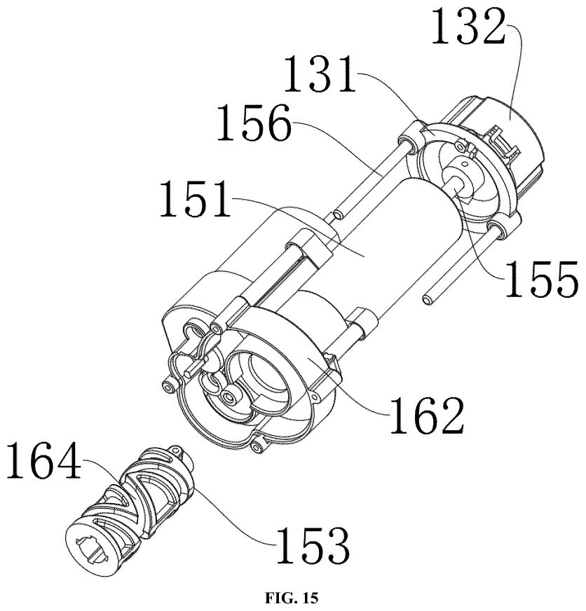

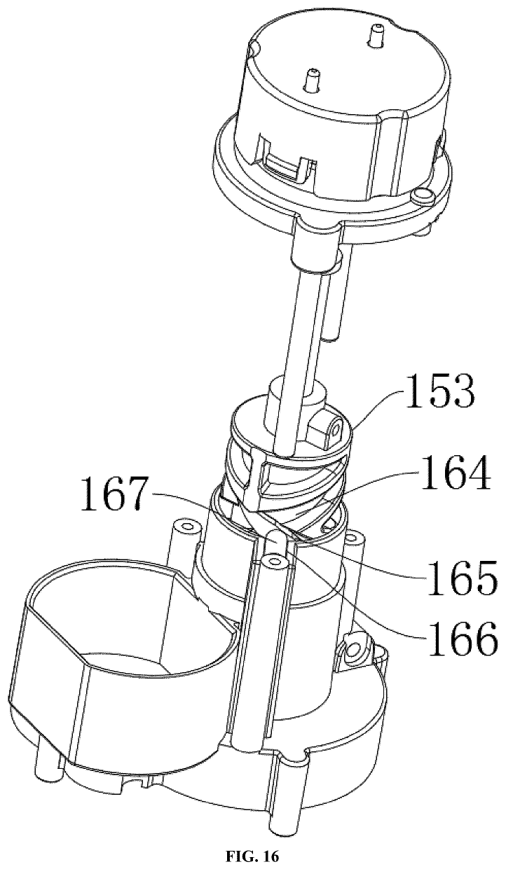

Preferably, as shown in FIGS. 14 and 15, a protective casing 162 radially extends from a mounting end of the fixed cylinder 151, the motor 157 is fixed at the protective casing 162, the rotating shaft and the gear set 154 of the motor 157 are both positioned in the protective casing 162, a protective cover 163 covers and is fixed at an opening of the protective casing 162, the gear set 154 comprises a driving gear 158, a first driven gear 159, a second driven gear 160 and a third driven gear 161, the driving gear 158 is engaged with the first driven gear 159, the first driven gear 159 and the second driven gear 160 are stacked and connected by a rotating shaft, the second driven gear 160 is engaged with the third driven gear 161, and a central part of the third driven gear 161 is connected to the driving end of the reciprocating screw 153. The protective casing 162 and the protective cover 163 are arranged to prevent the gears from being exposed and contaminated with dust, so that the gears are protected. The gear set 154 is arranged to provide a speed reduction effect for a driving force of the motor 157, the driving gear 158 and the driven gear 159 can be a helical gear or a spur gear, and the second driven gear 160 and the third driven gear 161 can be a spur gear.

Further, the fixed frame 19 is positioned in the mounting cavity 121, the positioning cylinder 18 and the fixed frame 19 are spliced and fixed through bolts, the motor 157 and the air extraction pump 16 are positioned between the positioning cylinder 18 and the fixed frame 19, and this structural configuration may be achieved; and the air extraction pump 16 and the protection casing 162 are fixed to the fixed frame 19.

Further, a flexible ring 10 is mounted at an opening of the first cylinder 11, the flexible ring 10 is made of silica gel, the outer wall of the flexible ring 10 abuts the inner wall of the first cylinder 11, and an end cover is buckled at the opening of the first cylinder 11.

The present invention is not limited to the above optimal implementations, and any other various products may be obtained by anyone in light of the present invention. However, no matter what change in shape or structure thereof is made, all technical solutions that are identical or similar to those of the present invention will fall within the protection scope of the present invention.

What is claimed is:

1. A multifunctional massage device, comprising a housing, wherein the housing is provided with a mounting cavity and a massage cavity, a mounting member, a driving device, an air extraction pump and an electric control valve are mounted in the mounting cavity, a mounting cylinder and a flexible massage cylinder are placed in the massage cavity, an opening of the flexible massage cylinder faces an opening of the massage cavity, and the flexible massage cylinder is mounted in a cylinder cavity of the mounting cylinder; an electric heating sheet is bonded on an inner wall of the cylinder cavity of the mounting cylinder, or a vibrator is embedded in the flexible massage cylinder; the driving device is mounted on the mounting member;
the driving device comprises a motor, a fixed cylinder, a gear set, a reciprocating screw, a telescopic member and a crescent arc guide member, the fixed cylinder is provided with a guide channel arranged along a length direction of the fixed cylinder, the reciprocating screw penetrates through the guide channel, a rotating shaft of the motor is in transmission connection with a driving end of the reciprocating screw through the gear set, a telescopic end of the reciprocating screw is fixedly connected to a telescopic shaft, a driving end of the telescopic shaft passes through a closed end of the fixed cylinder and then is connected to the telescopic member, two mutually crossed and communicated spiral grooves are formed on the reciprocating screw, the arc guide member is positioned in the spiral groove, and a shaft body of the arc guide member is rotatably mounted in a shaft hole on an inner wall of the guide channel;
the mounting member is provided with an input through hole and an air extraction through hole, an air extraction port of the air extraction pump is communicated with the air extraction through hole through a pipeline, an air inlet of the electric control valve is connected to and communicated with the input through hole through a pipeline, and a flexible ring is mounted at a port of the housing; and
a central mounting part of the mounting cylinder is mounted on the telescopic member, a conductive structure is provided between the telescopic member and the central mounting part of the mounting cylinder, the conductive structure is electrically connected to the vibrator or the electric heating sheet through a conductive wire, and the mounting cylinder is provided with a communicating port.

2. The multifunctional massage device according to claim 1, wherein the conductive structure comprises a first connecting body, a second connecting body and a third connecting body, the second connecting body is positioned between the first connecting body and the third connecting body, the first connecting body and the second connecting body are mounted on the telescopic member, the third connecting body is fixed on the central mounting part of the mounting cylinder, two first conductive parts arranged at intervals on the first connecting body respectively abut against two second conductive parts arranged at intervals on the second connecting body, two third conductive parts arranged at intervals on the second connecting body respectively abut against two fourth conductive parts on the third connecting body, the second conductive parts of the second connecting body are separately connected to two third conductive parts through a conductive circuit, two third conductive parts on the second connecting body are respectively a positive pole and a negative pole, two fourth conductive parts on the third connecting body are respectively connected to a positive electrode and a negative electrode of a motor of the vibrator, and the two first conductive parts on the first connecting body are both annular conductive contact pieces and are concentrically arranged.

3. The multifunctional massage device according to claim 2, wherein at least one fifth conductive part is further provided on the second connecting body, the fifth conductive parts of the second connecting body are separately connected to two third conductive parts through a conductive circuit, the third conductive part on the second connecting body is arranged opposite to the fourth conductive part, and the two fifth conductive parts respectively abut against two first conductive parts of the first connecting body.

4. The multifunctional massage device according to claim 3, wherein the first connecting body is provided with a sixth conductive part spaced apart from the first conductive part, the second connecting body is further provided with a seventh conductive part and an eighth conductive part, the seventh conductive part is connected to the eighth conductive part through a conductive circuit, the eighth conductive part is a positive pole, the third connecting body is provided with a ninth conductive part spaced apart from the fourth conductive part, the seventh conductive part abuts against the sixth conductive part, the eighth conductive part abuts against the ninth conductive part, the ninth conductive part and a fourth conductive part that abuts against the third conductive part of the negative pole of the third connecting body and that is on the third connecting body are respectively connected to the positive electrode and the negative electrode of the electric heating sheet, and the sixth conductive part is an annular conductive contact piece and is concentrically arranged with the first conductive part.

5. The multifunctional massage device according to claim 1, wherein the mounting member comprises a positioning cylinder, the fixed cylinder is positioned in a positioning channel of the positioning cylinder, and the telescopic member is provided with a guide rod that is inserted into a guide hole of the positioning cylinder.

6. The multifunctional massage device according to claim 2, wherein the telescopic member comprises a telescopic disk and a telescopic sleeve, the telescopic sleeve is sleeved on a positioning ring of the telescopic disk and forms a cavity, the first connecting body and the second connecting body are fixed in the cavity, the driving end of the telescopic shaft is connected to the telescopic disk or the telescopic sleeve after penetrating through the telescopic disk, a plurality of gaps are formed at a peripheral side of the telescopic sleeve at intervals, at least one elastic buckle is formed on the telescopic sleeve through the gaps, the central mounting part of the mounting cylinder is a sleeve body sleeved on the telescopic sleeve, at least one buckle groove is formed on an inner wall of the sleeve body, the elastic buckle is correspondingly buckled in the buckle groove, the third connecting body is fixed in the sleeve body, and the two third conductive parts arranged at intervals on the second connecting body abut against the two fourth conductive parts on the third connecting body after penetrating through the telescopic sleeve.

7. The multifunctional massage device according to claim 6, wherein at least one limiting protrusion is formed at an inner wall of the sleeve body, at least one limiting recess is formed at a peripheral side of the telescopic sleeve, and the limiting protrusion is correspondingly embedded in the limiting recess.

8. The multifunctional massage device according to claim 5, wherein a protective casing radially extends from a mounting end of the fixed cylinder, a protective cover covers and is fixed at an opening of the protective casing, the motor is fixed at the protective casing, the rotating shaft and the gear set of the motor are both positioned in the protective casing, a fixed frame is mounted in the mounting cavity, the fixed cylinder is spliced with the fixed frame, the motor and the air extraction pump are positioned between the positioning cylinder and the fixed frame, and the air extraction pump and the protective casing are fixed on the fixed frame.

9. The multifunctional massage device according to claim 8, wherein the gear set comprises a driving gear, a first driven gear, a second driven gear and a third driven gear, the driving gear is engaged with the first driven gear, the first driven gear and the second driven gear are stacked and connected by a rotating shaft, the second driven gear is engaged with the third driven gear, and a central part of the third driven gear is connected to the driving end of the reciprocating screw.

10. The multifunctional massage device according to claim 4, wherein the housing comprises a second cylinder and a first cylinder, the mounting cavity is arranged on the second cylinder, the massage cavity is arranged on the first cylinder, the second cylinder is connected to the first cylinder through the positioning cylinder, and the flexible ring is mounted at a port of the first cylinder.
